(19) United States
(12) Patent Application Publication
(10) Pub. No.: US 2025/0002874 A1
(43) Pub. Date: Jan. 2, 2025

(54) Method for Cell-Free Production of Unspecific Peroxygenases

(71) Applicant: Brandenburgische Technische Universitaet Cottbus-Senftenberg, Cottbus (DE)

(72) Inventors: Katrin Scheibner, Senftenberg (DE); Kai-Uwe Schmidtke, Senftenberg (DE); Marina Schramm, Atlanta, GA (US)

(21) Appl. No.: 18/688,601

(22) PCT Filed: Sep. 5, 2022
(86) PCT No.: PCT/EP2022/074636

§ 371 Date: Mar. 1, 2024

(30) Foreign Application Priority Data
Sep. 3, 2021 (DE) 10 2021 209 758.8
Dec. 17, 2021 (DE) 10 2021 214 582.5

(51) Int. Cl. C12N 9/08

(52) U.S. Cl. CPC C12N 9/0065; C12Y 111/02001

(57) ABSTRACT

The present invention relates to a process for the cell-free production of unspecific peroxygenases, preferably from fungi and/or genetically modified variants thereof, using eukaryotic cell extracts, preferably from fungi, in particular filamentous fungi, and its use.

### OBJECT OF THE INVENTION

The present invention relates to a process for the cell-free production of unspecific peroxygenases, preferably from fungi and/or genetically modified variants thereof, using eukaryotic cell extracts, preferably from fungi, in particular filamentous fungi, and its use.

### STATE OF THE ART

There is considerable interest in manufacturing processes for non-specific peroxygenases, i.e. enzymes with peroxidase and peroxygenase activity within the meaning of the present invention, which are summarized under EC number 1.11.2.1 [BRENDA database, as of September 2021] (hereinafter referred to as “UPO”), in order to be able to use them effectively as biocatalysts for the pharmaceutical and chemical industries, for example in the manufacture of pharmaceuticals, cosmetics, foodstuffs, adhesives, dyes or in sensor technology or biological assays.

UPOs belong to the heme thiolate proteins and selectively catalyze oxygen transfer reactions of hydroperoxides, preferably hydrogen peroxide, in organic molecules, including non-activated hydrocarbons, thereby generating hydroxylations and epoxidations. Compared to other oxygen transfer-catalyzing enzymes, UPOs work independently of electron donors, transport proteins and additional cofactors. The classic chloroperoxidase from Leptoxyphium fumago also belongs to this protein family.

The majority of fungal UPOs known to date belong to the subkingdom of the Dikarya, especially from the division of the Basidiomycota and Ascomycota. Phylogenetically, the UPO sequences are divided into two large protein families, called long and short UPOs. The UPO genes are identified by conserved amino acids that are essential for catalytic functionality. The PCP-EGD motif is typical for long UPOs, such as the UPO of Agrocybe aegerita (syn. Cyclocybe aegerita, gene APO1), while the PCP-EHD motif is more typical for short UPOs, such as the UPO of Chaetomium globosum (gene CHGG 00319; Hofrichter et al. 2015, Kiebist et al., 2017). For many of the putative UPO genes, the presence of signal peptides was predicted bioinformatically, which means that they belong to secreted proteins that are generally glycosylated. Furthermore, the UPOs from Agrocybe aegerita and Marasmius rotula, for example, are known to possess intra- and intermolecular disulfide bridges, respectively. Therefore, (many) UPOs are dependent on post-translational modifications for expression.

A selection of UPOs has already been used for the conversion of various pharmaceuticals and for the synthesis of fine and specialty chemicals (Hofrichter et al., 2020, Kiebist et al., 2019). However, there is a problem with the universal availability of UPOs. The discrepancy between the number of UPOs that can actually be produced and the number of bioinformatically annotated putative UPO genes from genome sequencing is large. This means that there is still no reliable access to the full spectrum of UPO-catalyzed reactions and therefore the full industrial potential of this enzyme class cannot be exploited.

No cell-free production of unspecific peroxygenases, especially using eukaryotic cell extracts, e.g. from fungi, is described in the state of the art.

In the state of the art, UPOs are synthesized homologously or heterologously in corresponding organisms. The first UPO was discovered in 2004 in the southern aegerita (Agrocybe aegerita) (Ullrich et al., 2004). Since then, further UPOs have been isolated from the wild types Coprinellus radians, Marasmius rotula, Chaetomium globosum and Marasmius wettsteinii, among others. The yields were between 10 and 450 mg protein/L. It takes several days to weeks to produce the UPOs, although finding suitable conditions to induce UPO production generally takes longer.

EP2468852B1 describes the isolation of a polypeptide with peroxygenase activity, but produced in cell-based systems.

WO2016207373A1 describes polypeptides with peroxygenase activity and their preparation, but in cell-based systems.

A disadvantage of the state of the art is that, despite bioinformatically identified UPO genes in the fungal genome, it is rarely possible to express and isolate the corresponding enzymes in these organisms. Therefore, putative UPOs cannot be produced by homologous production in sufficient diversity and yield and without great expenditure of time. A further disadvantage is that genetic engineering methods are not available for many wild types, so that the production of genetically modified UPO variants with desired altered properties (protein engineering) is not possible.

In the state of the art, the heterologous production of UPOs was first described in 2014 for AaeUPO in Saccharomyces cerevisiae and later extended as a tandem expression system with Pichia pastoris (Molina-Espeja et al., 2015).

WO2017081355A1 describes polypeptides with peroxygenase activity and their heterologous production, but in cell-based systems.

The first heterologous expression of UPOs in the bacterial system was achieved in 2018 with MroUPO in Escherichia coli. Subsequently, several variants and other UPOs such as Collariella virescens UPO were expressed.

EP3594332A1 describes a method for the heterologous expression of active peroxygenase from fungi and/or variants in bacterial cells, preferably Escherichia coli, but in cell-based systems.

A disadvantage of the state of the art for heterologous expression in eukaryotic cell systems is that it usually takes years before a UPO is successfully expressed and adapted. The yields are around 300 mg protein/L. Since the first description in 2014, only one UPO and its variants have been produced.

In the state of the art for heterologous expression in prokaryotic cell systems, the disadvantage is that the yields are in the one to lower two-digit mg/L range and the time from the production of a nucleic acid template with the genetic information for the UPO to the successful expression of the UPO is several days in the best case. In addition, there is generally no post-translational modification in prokaryotic cell systems, which means that the production of native UPOs is not possible.

The invention sets itself the task of producing native and genetically modified UPOs cell-free, preferably in less than 24 hours, and in this way providing new oxyfunctionalizing biocatalysts. The pure production process of the UPOs advantageously does not have to be carried out in laboratories with at least safety level 1 (according to DE-BioStoffV).

The biocatalysts can be used particularly effectively in the pharmaceutical industry (e.g. for the synthesis of active ingredients and drug metabolites) and chemical industry (e.g. for the synthesis of specialty and fine chemicals, some of which are chiral) as well as in sensor technologies or biological assays.

This task is solved by at least one patent claim.

### The Present Invention

The object of the present invention is a process for the cell-free production of unspecific peroxygenases from fungi and/or genetically modified variants thereof using eukaryotic cell extracts, preferably from fungi, in particular filamentous fungi.

With the aid of the process according to the invention, the universal production of unspecific peroxygenases in active form is possible in a batch or dialysis process with low process engineering and equipment costs.

The basis of the method according to the invention is cell-free protein synthesis with eukaryotic extracts. Cell-free protein synthesis has established itself as an efficient alternative to cell-based protein expression. The high-molecular components from cells required for protein synthesis are obtained from the cell extracts used and mixed with low-molecular components such as amino acids, energy-rich triphosphates (ATP, GTP) and various ions. Furthermore, functional microsomes are present in these extracts due to the production process of the cell extracts. These are vesicles of the endoplasmic reticulum, which are essential for the cell-free protein synthesis of secretory proteins (including many UPOs). Post-translational modifications such as N-glycosylation or disulfide bridge formation can take place in these microsomes. The endogenous mRNA in the eukaryotic cell extracts can be removed by various methods, in particular the addition of nucleases, especially RNases. Finally, the nucleic acid template is added for cell-free protein synthesis of the specific target protein, coding for one or more UPOs.

Therefore, the invention relates to a method for the production of unspecific peroxygenases (UPO) in cell-free systems using eukaryotic cell extracts, comprising the following steps:
i. Preparation of eukaryotic cell extracts for use in cell-free protein synthesis, ii. Provision of a nucleic acid template that contains the genetic information for the UPO, iii. Provision of further components which are necessary for the translation of proteins, in particular amino acids, energy-rich triphosphates, salts, RNA polymerase, or at least one or more components selected from the group consisting of amino acids, energy-rich triphosphates, salts, RNA polymerase, iv. Combining the cell extract from i.), the nucleic acid template from ii.) and the other components from iii.).

The advantages of the process according to the invention lie in the production speed of the UPOs of a few hours to less than two days, the high throughput with which many different UPOs can be produced in parallel, the variability with regard to changing the genetic information of the UPOs (introduction of mutations, protein engineering) and the openness of the system, which enables, for example, the possibility of adding exogenous components such as hemin. In addition, the production of potentially cytotoxic proteins is made possible and efficient labeling of proteins is facilitated. A further advantage of the process according to the invention is its universal applicability for the production of specific or native UPOs. Since cell extracts of filamentous fungi, which themselves carry (putative) UPO genes in the genome, are used for the cell-free system, it can be assumed that all factors necessary for UPO expression (e.g. chaperones) are present in the cell extracts. Therefore, new putative UPO genes can be added to the system in the form of a nucleic acid template without great effort and time-consuming adaptations and can therefore be produced cell-free.

In the context of the present invention, a “cell-free system” is understood to be one that does not require the integrity of a living cell and allows cell-free protein synthesis.

According to the invention, two methods are used for cell-free protein synthesis. In batch systems, protein synthesis takes place in a closed system in which all the necessary components are located. A continuous supply or removal of components is not possible, so that synthesis comes to a standstill once the components have been used up. In the dialysis process, a constant supply of reactants and removal of products is possible, which maintains the synthesis performance over a longer period of time.

Various translation systems are known, both from prokaryotic organisms such as extracts of Escherichia coli and eukaryotic organisms such as wheat germ extracts, rabbit reticulocyte extracts, insect cell extracts and CHO (Chinese Hamster Ovary) cell extracts.

The eukaryotic extract used according to the invention is not limited as long as it contains all the components necessary for the in vitro/ex vivo translation of the exogenous nucleic acid template for the synthesis of a non-specific peroxygenase.

The eukaryotic extracts preferred for the present invention originate from fungi, preferably filamentous fungi, particularly preferably from Aspergillus spp. and Neurospora spp, especially Aspergillus niger and Neurospora crassa.

In the particular embodiment, the fungal cell extracts used originate from Aspergillus niger and Neurospora crassa. The organisms are cultivated in liquid cultures, preferably between 50 mL and 30 L, particularly preferably between 100 mL and 1 L, especially in 200 mL medium.

The cultivation medium is composed of glucose and yeast extract, preferably between 5 and 50 g/L each, in the case of Neurospora crassa particularly preferably at 10 g/L, in the case of Aspergillus niger particularly preferably at 20 g/L.

The cultivation temperature of the filamentous fungi is preferably between 10° C. and 50° C., particularly preferably between 20° C. and 40° C., especially at 34° C. for Neurospora crassa and 30° C. for Aspergillus niger.

The cultivation time is preferably between 6 h and 96 h, particularly preferably between 12 h and 60 h, especially 48 h for Neurospora crassa and 24 h for Aspergillus niger. The biomass produced can be digested using various methods. In the special embodiment, the digestion was carried out using a high-pressure cell.

After harvesting, the respective mycelium is suspended in lysis buffer by filtration and repeated washing with mannitol buffer. The mycelium is digested at temperatures around 4° C. using high-pressure cell disruption at pressures between 1,000 and 20,000 psi, preferably at 5,000 psi for Neurospora crassa and 10,000 psi for Aspergillus niger. The digested mycelium is centrifuged several times at 5,000-10,000 g (preferably 6, 500 g) before use.

The endogenous mRNA of the crude lysate can be removed using various methods. In the specific example, a micrococcal nuclease was used.

The nucleic acid template, which contains the genetic information for the non-specific peroxygenase, can consist of DNA or RNA and be present in linear or cyclic form. The sequence used can be of the wild type or contain targeted or randomized mutations and can be used in the form of chimeras. The protein-coding nucleic acids are known from sequencing studies and are collected in databases which are continuously expanded so that suitable expression constructs with corresponding promoters (e.g. T7 RNA polymerase promoter) and regulatory sequences (e.g. 5′-untranslated region of a highly expressed gene) can be constructed and flexibly varied for selected UPOs,

According to the invention, the reaction mixture for the cell-free synthesis of UPOs contains, in addition to the eukaryotic cell extract and the selected nucleic acid template, further mostly low-molecular components such as amino acids and energy-rich triphosphates (ATP, GTP). Other additives necessary for synthesis or protein stability, such as hemin or detergents, may also be included. If the nucleic acid template added is DNA, a corresponding RNA polymerase, in particular T7 RNA polymerase, is added to the system in order to run the synthesis as a coupled transcription/translation. The reaction sets can be carried out in batch or dialysis mode. The reaction sets can be carried out for between 30 min and 48 h (preferably 4 h).

The cell-free UPOs produced according to the invention can be used to carry out single to high-throughput screenings with substrates that are relevant, for example for use in the pharmaceutical and chemical industries. The cell-free UPO can be used purified or unpurified.

The cell-free UPO proteins produced according to the invention can be isolated and purified using known protein chemistry methods such as hydrophobic interaction chromatography, ion exchange chromatography, size exclusion chromatography or with special tags via affinity chromatography.

UPOs according to the invention catalyze similar reactions as the P450 monooxygenases, in particular the incorporation of an oxygen atom into the substrate in the presence of a suitable oxidizing agent, preferably in buffered aqueous solutions. The catalyzed reactions comprise oxygen transfer reaction of hydroperoxides in organic molecules of the hydrocarbon type consisting of aromatic, aliphatic, linear, cyclic, and branched hydrocarbons as well as structural analogs. Preferred processes according to the invention are processes for the oxidative conversion of substrates, in particular hydroxylation and epoxidation, in order to obtain products with improved or desired properties, such as the synthesis of agrochemicals, herbicides, insecticides, medicaments, cosmetics, adhesives, dyes.

The only cosubstrate required for the oxyfunctionalization reaction is a hydroperoxide (R—OOH), preferably hydrogen peroxide (H₂O₂).

The UPOs produced by the present process are used in a low concentration of 0.01 U mL⁻¹ to 10 U mL⁻¹, whereby a concentration between 1 and 5 U mL⁻¹ is optimal for the oxyfunctionalization of organic compounds (1 unit converts 1 μmol of veratryl alcohol per minute).

In the present method, the concentration of the substrate is between 0.1 and 10 mM, with between 0.2 and 5 mM being preferred, particularly between 0.5 and 2 mM.

In a preferred embodiment, the process is carried out in aqueous, buffered solutions. Buffers such as phosphates or organic acids, preferably citric acid, can be added to the reaction mixture to stabilize the pH value. The buffer concentration is preferably between 1 mM and 100 mM, in particular between 10 and 20 mM. The reaction process is carried out at pH values of 3 to 10, preferably at 5 to 8, in particular at pH 7.

Organic solvents can be added to the reaction mixture to improve the solubility of the substrate. Preferred solvents are water-miscible solvents such as alcohols, acetone and acetonitrile. The concentration of the solvent is in the range of 1-90% (v/v), particularly preferably from 2-50% (v/v), especially from 5-30% (v/v).

Hydrogen peroxide (H₂O₂) is preferably used as the oxidizing agent. This means that cost-intensive cosubtrates, such as NADH or NADPH as electron donors, can be dispensed with in the present process. Additional electron transport proteins and regulatory proteins (flavin reductases, ferredoxins), as in the P450 system, are not required.

In reactions with UPOs produced by the process according to the invention, the cosubstrate can be dosed once, stepwise or continuously. The dosed H O₂₂ concentration is between 0.1 and 20 mM per hour, preferably between 0.5 mM and 5 mM, particularly between 1-3 mM per hour.

Alternatively, organic hydroperoxides (R—OOH, e.g. tert-butyl hydroperoxide), peroxycarboxylic acids (R—COOOH, e.g. meta-chloroperbenzoic acid) or hydrogen peroxide adducts (e.g. carbamide peroxide) can be used.

The use of UPOs enables an embodiment at normal pressure and temperatures of 4-40° C., particularly at 15-35° C., especially at 20-30° C.

The enzymatic conversion is usually completed within 24 hours, preferably within 5 minutes to 4 hours, particularly preferably in 30 minutes to 3 hours.

Reactions with UPOs produced by the process according to the invention can be carried out in a one-step reaction and the products obtained can be purified if necessary, for example by extraction, filtration, distillation, rectification, chromatography, treatment with ion exchangers, adsorbents or crystallization. Preferably, the products are isolated and purified by means of liquid-liquid extraction and chromatographic separation processes.

The invention will be explained in more detail below with reference to the examples and illustrations, without however limiting the invention to these examples and illustrations.

### EXAMPLES AND ILLUSTRATIONS

The present invention is explained in more detail below with reference to examples and figures. The examples include the cell-free production of a long UPO from Agrocybe aegerita (syn. Cyclocybe aegerita; AaeUPO for short), the cell-free production of a genetically modified variant of the first-mentioned UPO (AaeUPO PaDa-I), and the cell-free production of a short UPO from Marasmius rotula (MroUPO).

### Reference Example 1—Preparation of Cell Extracts

### 1) Cultivation of the Mushrooms

### Example 1 Aspergillus niger

Aspergillus niger (DSM 11167) was cultivated in 500 mL Erlenmeyer flasks, each containing 100 ml 2HA-MS medium (according to Nieland et al, 2021; but without antifoam agent). After inoculation with one drop of spore suspension each (3*10⁸ spores/mL glycerol), the flasks were incubated on the rotary shaker at 30° C. for 24 h at 150 rpm.

### Example 2 Neurospora crassa

Cultivation of Neurospora crassa (DSM 1257) was performed in 500 mL Erlenmeyer flasks, each containing 200 mL HA complete medium (Nieland and Stahmann, 2013; 10 g/L glucose and 10 g/L yeast extract). After inoculation with one drop of spore suspension each (107 spores/mL glycerol), the flasks were incubated on the rotary shaker at 34° C. for 48 h at 120 rpm.

### 2) Preparation of Cell Extracts for Cell-Free Protein Synthesis

### Example 1 Aspergillus niger

The mycelium cultivated as in (1) was harvested by sucking with a Büchner funnel (equipped with VWR filter paper 413) and washing twice with 50 mL of 4° C. cold mannitol buffer A (Hodgman and Jewett, 2013). A. niger biomass of 5.14 g (fresh weight) was harvested from 200 mL of medium. The mycelium was then suspended with 1.5 mL of lysis buffer A (Hodgman and Jewett, 2013) per g of fresh weight and subsequently digested at 4° C. using high-pressure cell disruption (HTU Digi-F-Press, manufacturer G. Heinemann Ultraschall-und Labortechnik) at 10,000 psi. All steps were carried out as quickly as possible and as far as possible on ice. The digested mycelium was centrifuged at 4° C. and 6500 g for 5 minutes. The same procedure was repeated with the supernatant.

To remove the endogenous mRNA from the supernatant, the crude lysate was then pretreated at room temperature for 10 min with Micrococcal Nuclease (Thermo Scientific) according to Hodgman and Jewett, 2013. The treated extract can be aliquoted with liquid nitrogen, flash frozen and stored at −80° C.

### Example 2 Neurospora crassa

The mycelium cultivated as in (1) was harvested by nutsching with a Büchner funnel (equipped with VWR filter paper 417) and washing twice with 50 mL of 4° C. cold mannitol buffer A (Hodgman and Jewett, 2013). From 200 mL medium, 6 g biomass (fresh weight) of N. crassa was harvested. The mycelium was then resuspended with 1.5 mL lysis buffer A (Hodgman and Jewett, 2013) per g fresh weight and subsequently digested at 4° C. using high-pressure cell disruption (HTU Digi-F-Press, manufacturer G. Heinemann Ultraschall-und Labortechnik) at 5,000 Psi. The further procedure was analogous to example 1 Aspergillus niger.

### Reference Example 2—Production of Nucleic Acid Template

### Example 1 Production of mRNA AaeUPO-his

### 1) Preparation of a DNA Template for In Vitro Transcription

A plasmid was generated consisting of the pYES2 vector and the Saccharomyces cerevisiae codon optimized sequence for the wild-type UPO AaeUPO with signal and propeptide and a C-terminal His₆ tag (synthesized by GeneArt). Upstream of the AaeUPO gene is a promoter for the T7 RNA polymerase. For run-off transcription, the plasmid was linearized with the restriction endonuclease BstEII and subsequently purified by chromatography.

### 2) In Vitro Transcription

The DNA fragment with T7 promoter and AaeUPO His gene prepared in section (1) above was used as a template in an in vitro transcription reaction. The in vitro transcription was performed according to the instructions of the manufacturer New England Biolabs using the HiScribe™ T7 ARCA mRNA Kit (with tailing).

The RNA formed was purified by chromatography.

In this way, 26 μg of 5′-capped, polyadenylated AaeUPO-His mRNA was obtained, verified by measuring the absorbance at 260 nm.

### Example 2 Production of mRNA AaeUPO PaDa-I-his

### 1) Preparation of a DNA Template for In Vitro Transcription

A plasmid was generated consisting of the pYES-DEST52 vector and the Saccharomyces cerevisiae codon-optimized sequence for the UPO mutant AaeUPO PaDa-I with signal and propeptide (Molina-Espeja et al., 2015; synthesized by GeneArt). Upstream of the AaeUPO PaDa-I gene is a promoter for the T7 RNA polymerase. By PCR (98° C., 10 s, 63° C., 20 s and 72° C., 35 s over 30 cycles), an AaeUPO PaDa-I variant with C-terminal His₆ tag was produced using the aforementioned plasmid, a primer (T7-PaDa_fw) with a base sequence shown in SEQ ID No. 1 and a primer (6HisPaDa_rev) with a base sequence shown in SEQ ID No. 2. The DNA fragment was purified by chromatography.

### 2) In Vitro Transcription and Polyadenylation

The DNA fragment with T7 promoter and AaeUPO PaDa-I-His gene prepared in section (1) above was used as a template in an in vitro transcription reaction. The in vitro transcription was performed according to the manufacturer's instructions New England Biolabs using the HiScribe™ T7 High Yield RNA Synthesis Kit. The transcription reaction was incubated for 2.5 h at 37° C.

The RNA formed was purified by chromatography.

For polyadenylation at the 3′-end of the RNA described above, E. coli poly (A) polymerase, 1×E. coli poly (A) polymerase reaction buffer, 1 mM ATP and 53 μg of AaeUPO PaDa-I-His RNA were used according to the instructions of the manufacturer New England Biolabs. The RNA formed was purified by chromatography. In this way, 73 μg of polyadenylated AaeUPO PaDa-I-His mRNA was obtained, verified by absorbance measurement at 260 nm.

### Example 3 Production of mRNA MroUPO-his

### 1) Preparation of a DNA Template for In Vitro Transcription

A plasmid was generated consisting of the pYES2 vector and the Saccharomyces cerevisiae codon optimized sequence for the wild type UPO MroUPO with signal peptide and a C-terminal His₆ tag (synthesized by GeneArt). Upstream of the MroUPO gene is a promoter for the T7 RNA polymerase. For run-off transcription, the plasmid was linearized with the restriction endonuclease BstEII and subsequently purified by chromatography.

### 2) In Vitro Transcription

The DNA fragment with T7 promoter and MroUPO-His gene prepared in section (1) above was used as a template in an in vitro transcription reaction. The in vitro transcription was performed according to the instructions of the manufacturer New England Biolabs using the HiScribe™ T7 ARCA mRNA Kit (with tailing).

The RNA formed was purified by chromatography.

In this way, 30 μg of 5′-capped, polyadenylated MroUPO-His mRNA was obtained, verified by measuring the absorbance at 260 nm.

### Reference Example 3—Production of UPO in a Cell-Free System

For the cell-free preparation of UPO, the cell extracts prepared according to reference example 1 example 1 Aspergillus niger and example 2 Neurospora crassa and the cell extracts prepared according to reference example 2 example 1 5′-Cap/Poly (A) AaeUPO-His, Example 2 Poly (A) AaeUPO PaDa-I-His and Example 3 5′-Cap/Poly (A) MroUPO-His and Poly (A) AaeUPO PaDa-I mRNA without C-terminal His₆ tag were used. For the latter mRNA, the plasmid listed in reference example 2 example 2 was linearized and purified using the restriction endonuclease PmeI for run-off transcription. Subsequent in vitro transcription and polyadenylation was performed as described in reference example 2 example 2.

### [Composition of the Cell-Free Translation Reactions]

25% translation mix, consisting of: Energy mix (80 mM HEPES; 4 mM ATP; 0.4 mM GTP; 8 mM DTT; 80 mM creatine phosphate); 200 mM potassium acetate; 4.8 mM magnesium acetate; 0.24 mg/ml tRNA from yeast; amino acid mixture (each amino acid 0.08 mM); 0.24 U/μl creatine phosphokinase; 3 U/μl RNase inhibitor (manufacturer applied biosystems)
28 nM mRNA
50% cell extract
DEPC-treated water (manufacturer Carl Roth)

The incubation was carried out at 18° C. for 270 min.

### Experimental Example

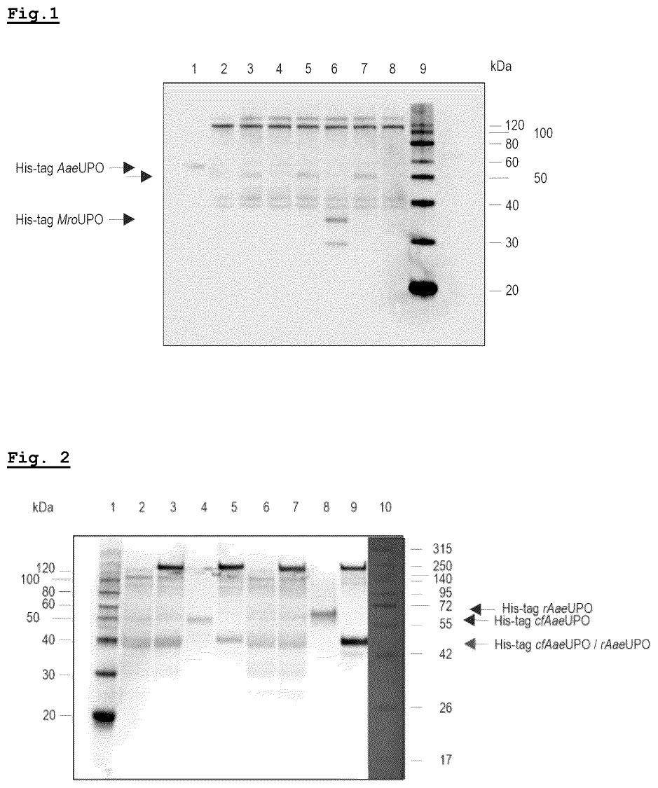

The production of the wild-type UPO AaeUPO, the AaeUPO variant PaDa-I with His₆ tag and the MroUPO according to reference example 3 was detected by SDS-polyacrylamide gel electrophoresis and subsequent Western blotting. The protein concentrations in the translation reactions without mRNA, with AaeUPO-His mRNA, with AaeUPO PaDa-I-His mRNA or AaeUPO PaDa-I mRNA without C-terminal His₆ tag and with MroUPO-His mRNA were determined by BCA assay. From each reaction mixture, 20 μg protein was applied to a 10% BisTris gel. Electrophoresis was performed with an MES buffer. The proteins were transferred to a PVDF membrane in a semidry blot procedure. The primary antibody against the His₆ tag was a monoclonal mouse 6x-His tag antibody from Invitrogen. Detection was carried out using an anti-mouse secondary antibody to which a horseradish peroxidase was coupled, whose catalytic activity generates a chemiluminescent signal (FIG. 1).

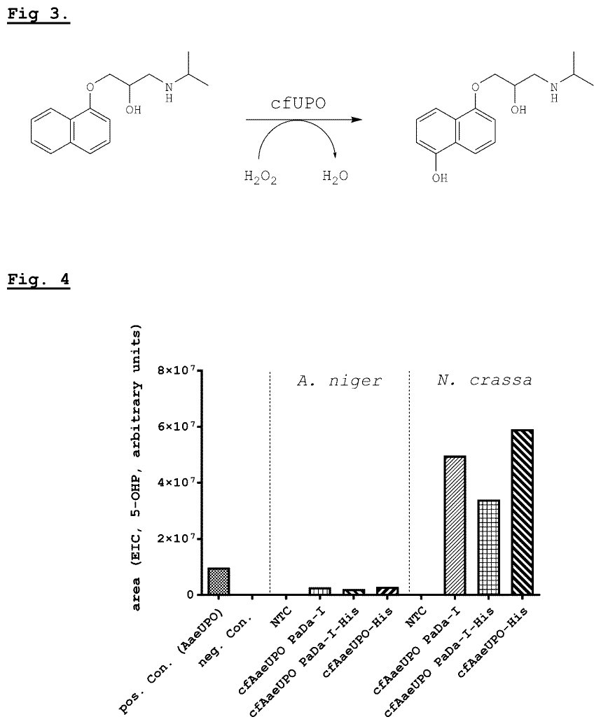

The localization of the UPOs in the microsomes was tested by fractionation of the cell-free reaction mixtures by centrifugation at 16,000 g, 4° C. for 10 min. The supernatants were transferred to new reaction tubes and the pellet containing the microsomes was resuspended in DEPC-treated water with the same volume as the supernatant. The presence of glycosylation on the cell-free UPOs was tested by deglycosylation with the protein Deglycosylation Mix II from New England Biolabs. The untreated and deglycosylated fractions were analyzed by Western blot as described above (FIG. 2).

### Reference Example 4—Oxyfunctionalization of Propranolol

The selective oxyfunctionalization of propranolol to 5-hydroxypropranolol (5-OHP) was carried out with cell-free UPO (FIG. 2). For the enzymatic synthesis of 5-OHP, 20 μL reaction medium of the cell-free UPO synthesis was added to 180 μL reaction solution in a 200 μL preparation. The reaction solution contained the following components: 800 UM propranolol (hydrochloride), 1 mM hydrogen peroxide, 5 mM ascorbate and 20 mM phosphate buffer (pH 7.0). The reaction mixture was incubated for 5 min at 25° C. and 800 rpm in a thermal shaker. Subsequently, the reaction mixtures were stopped by adding 200 μl acetonitrile (−20° C.) and centrifuged for 10 min. The supernatants were analyzed by HPLC-MS under the following conditions:

Chromatographic separation for the LC-MS experiments was performed using a Thermo Scientific Vanquish Flex Quaternary UHPLC system (Thermo Fisher Scientific, Waltham, MA, USA) with a Kinetex® column (C18, 5 μm, 100 Å, 150×2.1 mm, Phenomenex). The injection volume was 1 μL and the column was eluted at a flow rate of 0.5 mL/min and 40° C. with two mobile phases A (diH2O, 0.1% formic acid) and B (acetonitrile, 0.1% formic acid) and the following gradient: 0 min 10% B; 1 min, 10% B; 6 min, 80% B; 7 min, 80% B; 7.1 min, 10% B; 10 min, 10% B.

MS and MS-spectra were recorded using a Thermo Scientific Q Exactive Plus Quadrupole Orbitrap mass spectrometer (Thermo Electron, Waltham, MA, USA) coupled to a heated electrospray ionization source in positive mode ([M+H]⁺). The operating parameters were as follows: The sheath gas and auxiliary gas flow rates were 60 and 15 (arbitrary unit), respectively; the spray voltage was 4.0 kV; the capillary and auxiliary gas heater temperatures were 320° C. and 400° C., respectively; the high-resolution MS was operated in full-scan mode with a mass range of m/z 150-1,500 at a resolution of 70,000 (m/z 200). The MS-data with a resolution of 35, 000 were obtained in parallel reaction monitoring (PRM) mode, triggered by a list of the preselected intentions of propranolol and 5-hydroxypropranolol (C H₁₆₂₂ NO₂⁺ & C H₁₆₂₂ NO₃⁺). The collision energy was CE15.

The detected activities of the UPOs cfAaeUPO, cfAaeUPO PaDa-I and cfAaeUPO PaDa-I-His produced cell-free in reference example 3 are shown in FIG. 3.

### Reference Example 5—Substrate Screening with Unpurified Cell-Free UPO

The enzymatic conversion of analytical UPO substrates (veratryl alcohol; 2, 2′-azino-bis (3-ethylbenzothiazoline-6-sulfonic acid [ABTS]; 5-nitrobenzodioxole [NBD]; naphthalene) and pharmaceuticals (propranolol, diclofenac and clopidogrel) was carried out with unpurified, cell-free UPO.

For the reactions with the analytical substrates, 10 μL reaction medium of the cell-free UPO synthesis was added to 190 μL reaction solution in a 200 μL preparation. The reaction solution contained the following components: 5 mM veratryl alcohol or 0.6 mM ABTS or 0.5 mM NBD or 1 mM naphthalene, 2 mM hydrogen peroxide and 50 mM phosphate buffer (pH 7.0 or pH 4.5 for ABTS). The products formed from the analytical substrates were measured using a microplate photometer (CLARIOstar Plus, BMG Labtech, Ortenberg, Germany): Conversion of veratryl alcohol to veratral aldehyde (ε310=9,300 M-1 cm-1); formation of ABTS radical (ε420=36,000 M-1 cm-1), conversion of NBD to 5-nitrocatechol (8425=9,700 M-1 cm-1) and conversion of naphthalene to 1-naphthtol (£303=9,700 M-1 cm-1). The reaction kinetics were measured over 30 sec at room temperature.

The reactions with the pharmaceuticals and the detection of the reaction products (FIG. 4) were carried out as described in reference example 4 for propranolol.

The detected activities of the AaeUPO-His and MroUPO-His produced cell-free with N. crassa cell extract in reference example 3 are listed in Table 1. The enzyme activities are given in U/L, where 1 U corresponds to the formation of 1 μmol product per min.

TABLE 1
The table shows the enzyme activity of cell-free produced AaeUPO-
His and cell-free produced MroUPO-His with different substrates.

| | Enzyme activity | | |
|---|---|---|---|
| Substrate | | AaeUPO-His | MroUPO-His |

| | | | |
|---|---|---|---|
| Veratryl alcohol | 100 | mU/mL | n.b. |
| ABTS | 229 | mU/mL | n.b. |
| 5- | 93 | mU/mL | n.b. |
| Naphthalene | 212 | mU/mL | n.b. |
| Propranolol | 9972 | μU/mL | 300 μU/mL |
| Diclofenac | 4875 | μU/mL | n.b. |
| Clopidogrel | 2616 | μU/mL | 813 μU/mL |

n.b.—not determined

### Primer Sequences (PCR)

SEQ ID No. 1
5′-AGCAGCTGTAATACGACTCACTATAGGG-3′
SEQ ID No. 2
5′-TTTTTTTTCTCAATGGTGATGATGATGATGATCTACCATATGGAAA
AACTTGAG-3′

### Descriptions of the Illustrations

FIG. 1 Anti-His-tag Western blot analysis of cell-free and cell-derived wild-type UPO AaeUPO-His, the UPO mutant AaeUPO PaDa-I with C-terminal His₆-tag and MroUPO. (1) Ni-NTA purified AaeUPO PaDa-I-His from S. cerevisiae culture supernatant (2)-(8) cell-free protein synthesis: (2) control without mRNA (3) AaeUPO PaDa-I-His mRNA (4) internal control (5) AaeUPO PaDa-I mRNA-His (6) MroUPO-His mRNA (7) wild-type UPO AaeUPO-His mRNA (8) control without mRNA (9) MagicMark™ XP Western Protein.

FIG. 2 Anti-His-tag Western blot analysis of cell-free and cell-derived wild-type UPO AaeUPO-His for localization and glycosylation analysis. (1) MagicMark™ XP Western Protein; supernatant AaeUPO-His (2) untreated, (3) deglycosylated; pellet AaeUPO-His (4) untreated, (5) deglycosylated; control preparation without mRNA (6) untreated, deglycosylated (7); Ni-NTA purified AaeUPO PaDa-I-His from S. cerevisiae culture supernatant (8) untreated, (9) deglycosylated.

FIG. 3 Selective oxyfunctionalization of propranolol by cell-free UPOs (cfUPO).

FIG. 4 The diagram shows the peak area intensities in the extracted ion chromatogram (EIC) for 5-hydroxypropranolol (C H₁₆₂₂ NO₃⁺). The cell-free reaction approaches were used as in reference example 4 for the conversion of propranolol. As a positive control, 50 mU/mL (veratryl alcohol) AaeUPO was used.

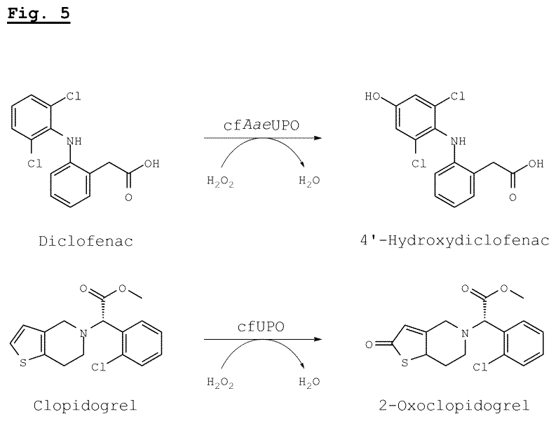

FIG. 5 The figure shows the oxyfunctionalization of diclofenac and clopidogrel by cell-free UPO (cfUPO) and cell-free AaeUPO (cfAaeUPO), respectively.

1. A method for the production of unspecific peroxygenases (UPO) in cell-free systems using eukaryotic cell extracts, comprising the following steps:
i) preparing eukaryotic cell extracts for use in cell-free protein synthesis;
ii) providing a nucleic acid template that contains the genetic information for the UPO;
iii) providing further components necessary for the translation of proteins, in particular amino acids, energy-rich triphosphates, salts, RNA polymerase; and
iv) combining the cell extract from i), the nucleic acid template from ii) and the other components from iii).

2. The method according to claim 1, wherein the eukaryotic cell extracts originate from fungi.

3. The method according to claim 2, wherein the fungal cell extracts originate from filamentous fungi, in particular of the genera Aspergillus and Neurospora, in particular of the species Aspergillus niger and Neurospora crassa.

4. The method according to claim 1, wherein the cell extracts are obtained by mechanical digestion, in particular high-pressure cell digestion.

5. The method according to claim 1, wherein the cell extract contains functional microsomes.

6. The method according to claim 1, wherein the nucleic acid template encodes a UPO of class EC 1.11.2.1.

7. The method according to claim 6, wherein the UPO coding nucleic acid template is linear or circular DNA or mRNA.

8. The method according to claim 1, wherein the translated UPO contains targeted or random mutations.

9. The method according to claim 1, wherein the translated UPO contains targeted tags.

10. The method according to claim 1, wherein the translated UPO contains targeted markers.

11. The method according to claim 1, wherein the UPO produced in the cell-free system is used for screening with low molecular weight organic compounds; cosmetics; food; adhesives; dyes; sensors; or biological assays.

12. The method according to claim 1, wherein the translated UPO is isolated from the reaction medium.

13. The method according to claim 1, wherein the UPOs produced in the cell-free system are used for organic syntheses; bioremediation processes; the production of pharmaceuticals; cosmetics; foods; adhesives; dyes; sensors; or biological assays.

14. A method of using the UPOs produced in the cell-free system according to claim 1 for the oxidative conversion of substrates.


SEQUENCE LISTING (2 sequences)

SEQ ID NO: 1 - DNA, 28 bp, synthetic construct
  source 1..28: mol_type = other DNA
agcagctgtaatacgactcactataggg

SEQ ID NO: 2 - DNA, 56 bp, synthetic construct
  source 1..56: mol_type = other DNA
ttttttttctcaatggtgatggtgatgatgatctctaccatatggaaaaacttgag